(19) United States
(12) Patent Application Publication
(10) Pub. No.: US 2008/0091019 A1
(43) Pub. Date: Apr. 17, 2008

(54) Polymorphic form B2 of Ziprasidone base

(75) Inventors: Judith Aronhime, Rehovot (IL); Marioara Mendelovici, Rehovot (IL); Tamas Koltai, Natania (IL); Rinat Moshkovits-Kaptsan, Raanana (IL); Tamar Nidam, Yehud (IL)

Correspondence Address: KENYON & KENYON LLP, ONE BROADWAY, NEW YORK, NY, 10004, US

(73) Assignee: Teva Pharmaceuticals USA, Inc.

(21) Appl. No.: 11/999,369

(22) Filed: Dec. 4, 2007

Related U.S. Application Data
(62) Division of application No. 11/018,489, filed on Dec. 20, 2004.
(60) Provisional application No. 60/531,244, filed on Dec. 18, 2003.

(51) Int. Cl. C07D 417/14

(52) U.S. Cl. 544/368

(57) ABSTRACT

Provided is a crystalline form of ziprasidone base and processes for its preparation.

### CROSS-REFERENCE TO RELATED APPLICATIONS

This application is a divisional of U.S. patent application Ser. No. 11/018,489, filed Dec. 20, 2004, claims the benefit of U.S. provisional application No. 60/531,244, filed Dec. 18, 2003, the content of all of which is incorporated herein.

### FIELD OF THE INVENTION

The present invention relates to the solid state chemistry of ziprasidone.

### BACKGROUND OF THE INVENTION

Ziprasidone is an antipsychotic agent that is chemically unrelated to phenothiazine or butyrophenone antipsychotic agents. Ziprasidone has the following structure:

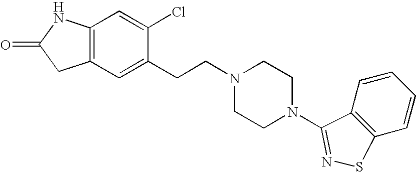

The preparation of ziprasidone base is disclosed in U.S. Pat. No. 4,831,031 (example 16) and U.S. Pat. No. 5,312,925. A process for preparation of ziprasidone HCl monohydrate having a mean particle size equal to or less than about 85 microns is also disclosed in U.S. Pat. No. 6,150,366 and EP 0 965 343 A2.

Ziprasidone has been marketed under the name GEODON as an oral capsule and as an injectable drug. GEODON capsules contain the monohydrate hydrochloride salt of ziprasidone, and come in 20, 40, 60 and 80 mg dosage forms. GEODON for injection contains a lyophilized form of ziprasidone mesylate trihydrate, and contains 20 mg base equivalent of ziprasidone. The mesylate salts of ziprasidone, including monohydrate and trihydrate, are disclosed in U.S. Pat. Nos. 6,110,918 and 5,245,765.

The present invention relates to the solid state physical properties of ziprasidone base. These properties can be influenced by controlling the conditions under which ziprasidone base or HCl is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. These conformational and orientational factors in turn result in particular intramolecular interactions and intermolecular interactions with adjacent molecules that influence the macroscopic properties of the bulk compound. A particular polymorphic form may give rise to distinct spectroscopic properties that may be detectable by powder X-ray diffraction, solid state ¹³C NMR spectrometry and infrared spectrometry. The polymorphic form may also give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others.

Ziprasidone HCl hemihydrate is disclosed in U.S. Pat. No. 4,831,031, Example 16 (column 13, line 13). Ziprasidone HCl monohydrate is disclosed in U.S. Pat. No. 5,312,925 and EP 0 586 181 A1. The monohydrate is characterized by XRD, IR and water content. It is reported that the water content of the monohydrate ranges from 3.8 to 4.5% by weight. The ziprasidone HCl monohydrate is prepared from ziprasidone base anhydrous.

Ziprasidone HCl is usually prepared from ziprasidone base, and the ziprasidone base used may affect the quality of the hydrochloride salt. Ziprasidone base in the solid state is disclosed in U.S. Pat. No. 5,338,846. In the '846 patent, ziprasidone base is characterized by its NMR spectrum. In example 1 of U.S. Pat. No. 5,206,366 ziprasidone base is also obtained. The base is characterized by NMR, thin layer chromatography and a melting point of 218-220 EC. In WO 03/070246 ziprasidone base is obtained from tetrahydrofuran. The product is not otherwise characterized. Ziprasidone base is also obtained in U.S. Pat. No. 5,312,925. The Form obtained in the art is labeled herein Form B of ziprasidone base.

Ziprasidone base Form B is characterized by X-Ray peaks at 12.1, 15.2, 16.3, 18.4, 25.0 degrees 2 theta and is further characterized by XRD peaks at 5.2, 10.4, 11.3, 13.1, 21.1, 22.1. The ziprasidone free base has a DSC thermogram in which 17 and 120 J/g endothermic peaks can be seen at 92 and 220° C. The first corresponds to dehydration, the second to melting of the ziprasidone free base. The water content of the sample of the base is about 1.2% by weight. The Loss on Drying by TGA is about 2.1% by weight.

US2004/152711 provides additional crystalline forms of ziprasidone HCl and base.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

In addition to allowing for improved formulations, a new polymorphic form may be used for calibration of XRD, FTIR or DSC instruments. The polymorphic form may further help in purification of an active pharmaceutical ingredient. In the event of metastability, a metastable polymorphic form may be used to prepare a more stable polymorph. Hence, discovery of new polymorphic forms and new processes help in advancing a formulation scientist in preparation of ziprasidone as an active pharmaceutical ingredient in a formulation.

There is a need in the art for additional polymorphic forms of ziprasidone base.

### SUMMARY OF THE INVENTION

In one aspect, the present invention provides for a crystalline form of ziprasidone base having an X-Ray powder diffraction pattern with peaks at 9.4, 13.7, 14.5, 14.9, 18.1, 20.2, 22.8±0.2 degrees 2 theta, labeled herein as Form B2.

In another aspect, the present invention provides a process for preparing the crystalline form B2 comprising:
a) reacting a salt of ziprasidone with a base in a reaction mixture containing water, and optionally a water-miscible organic co-solvent, to obtain the crystalline form of ziprasidone; and
b) recovering the crystalline form.

In another aspect, the present invention provides a process for preparing pharmaceutically acceptable salt of ziprasidone comprising:
a) reacting a salt of ziprasidone with a base in a reaction mixture containing water, and optionally a water-miscible organic co-solvent, to obtain the crystalline form of ziprasidone B2;
b) converting the crystalline form to a pharmaceutically acceptable salt of ziprasidone; and
c) recovering the pharmaceutically acceptable salt.

In another aspect, the present invention provides a process for preparing ziprasidone HCl comprising reacting HCl with crystalline ziprasidone base of B2 to obtain ziprasidone HCl, and recovering the ziprasidone HCl.

In another aspect, the present invention provides a process for preparing pharmaceutically acceptable salt of ziprasidone comprising:
a) reacting a salt of ziprasidone with a base, to obtain the crystalline form of ziprasidone B2;
b) slurrying the crystalline form obtained in a C₁ to C₄ alcohol;
c) combining the crystalline form with an acid to obtain a pharmaceutically acceptable salt of ziprasidone; and
d) recovering the pharmaceutically acceptable salt.

In another aspect, the present invention provides a process for preparing ziprasidone base characterized by an X-Ray diffraction-pattern with peaks at 12.1, 15.2, 16.3, 18.4,25.0, 5.2, 10.4, 11.3, 13.1, 21.1, 22.1±0.2 degrees 2 theta (Form B) comprising slurrying ziprasidone base B2 in an aprotic solvent to obtain the ziprasidone base and recovering the obtained ziprasidone base.

In another aspect, the present invention provides a process for preparing pharmaceutically acceptable salt of ziprasidone comprising:
a) slurrying ziprasidone base B2 in an aprotic solvent to obtain ziprasidone base characterized by an X-Ray diffraction pattern with peaks at 12.1, 15.2, 16.3, 18.4, 25.0, 5.2, 10.4, 11.3, 13.1, 21.1, 22.1±0.2 degrees 2 theta (Form B);
b) converting ziprasidone base to a pharmaceutically acceptable salt of ziprasidone; and
c) recovering the pharmaceutically acceptable salt.

In another aspect, the present invention provides a process for preparing pharmaceutically acceptable salt of ziprasidone comprising:
a) slurrying ziprasidone base B2 in an aprotic solvent to obtain ziprasidone base characterized by an X-Ray diffraction pattern with peaks at 12.1, 15.2, 16.3, 18.4,25.0,5.2, 10.4, 11.3, 13.1,21.1,22.1±0.2 degrees 2 theta (Form B);
b) slurrying the ziprasidone base obtained in step a) in a C₁ to C₄ alcohol;
c) combining the slurry with an acid to obtain a pharmaceutically acceptable salt of ziprasidone; and
d) recovering the pharmaceutically acceptable salt.

In another aspect, the present invention provides a process for preparing ziprasidone HCl having an X-Ray diffraction pattern having peaks at 10.9, 17.4 and 19.1±0.2 degrees 2 theta (Form A) comprising:
a) combining HCl with a slurry of ziprasidone base in a mixture of water and a water miscible solvent to obtain the crystalline form; and
b) recovering the crystalline form.

In another aspect, the present invention provides a process for preparing ziprasidone HCl monohydrate (form M) comprising precipitating the crystalline form from a solution of ziprasidone base in a solvent selected from THF, methanol, DMA, acetic acid and mixtures thereof by combining HCl with the solution and recovering the crystalline form, wherein ziprasidone base B2 is used to prepare the solution.

In another aspect, the present invention provides a process for preparing ziprasidone HCl hemihydrate comprising combining a solution of HCl with a slurry made from ziprasidone base B2 in a solvent selected from C₂-C₄ alcohols.

In another aspect, the present invention provides a process for preparing ziprasidone HCl monohydrate (form M) comprising further converting the ziprasidone HCl hemihydrate to ziprasidone HCl monohydrate by slurrying in water and recovering the monohydrate.

In another aspect, the present invention provides a process for preparing ziprasidone HCl anhydrous comprising combining a solution of HCl with a slurry made from ziprasidone base B2 in methanol, and recovering the anhydrous form.

In another aspect, the present invention provides a process for preparing ziprasidone HCl anhydrous comprising combining gaseous HCl with a slurry made from ziprasidone base B2 in C₁ to C₄ alcohols, and recovering the anhydrous form.

In another aspect, the present invention provides a process for preparing a crystalline ziprasidone HCl characterized by a powder XRD pattern with peaks at 9.1, 19.1, 25.7, 26.3, 26.9±0.2 degrees 2 theta (form J) comprising slurrying ziprasidone base B2 in a C₅ to C₁₂ aromatic or aliphatic hydrocarbon.

In another aspect, the present invention provides a process for preparing ziprasidone base having an X-Ray diffraction pattern with peaks at 12.1, 15.2, 16.3, 18.4, 25.0, 5.2, 10.4, 11.3, 13.1, 21.1, 22.1±0.2 degrees 2 theta (Form B) comprising heating ziprasidone base B2 to obtain ziprasidone base.

In another aspect, the present invention provides a process for preparing pharmaceutically acceptable salt of ziprasidone comprising:
a) heating ziprasidone base B2 to obtain ziprasidone base having an X-Ray diffraction pattern with peaks at 12.1, 15.2, 16.3, 18.4, 25.0, 5.2, 10.4, 11.3, 13.1, 21.1, 22.1±0.2 degrees 2 theta (Form B); and
b) converting ziprasidone base to a pharmaceutically acceptable salt of ziprasidone; and
c) recovering the pharmaceutically acceptable salt.

In another aspect, the present invention provides a process for preparing ziprasidone base having an X-Ray diffraction pattern with peaks at 12.1, 15.2, 16.3, 18.4, 25.0, 5.2, 10.4, 11.3, 13.1, 21.1, 22.1±0.2 degrees 2 theta (Form B) comprising combining an anti-solvent with a solution of ziprasidone base in tetrahydrofuran to precipitate the crystalline form and recovering the crystalline form, wherein the solution is prepared with ziprasidone base B2.

In another aspect, the present invention provides a process for preparing a pharmaceutically acceptable salt of ziprasidone comprising:
a) combining an anti-solvent with a solution of ziprasidone base in tetrahydrofuran to precipitate the crystalline form of ziprasidone base having an X-Ray diffraction pattern with peaks at 12.1, 15.2, 16.3, 18.4, 25.0, 5.2, 10.4, 11.3, 13.1, 21.1, 22.1±0.2 degrees 2 theta (Form B);
b) converting the crystalline form to a pharmaceutically acceptable salt; and
c) recovering the pharmaceutically acceptable salt.

In another aspect, the present invention provides a process for preparing ziprasidone base having an X-Ray diffraction pattern with peaks at 12.1, 15.2, 16.3, 18.4, 25.0, 5.2, 10.4, 11.3, 13.1, 21.1, 22.1±0.2 degrees 2 theta (Form B) comprising slurrying ziprasidone HCl in water in the presence of a base, followed by washing with methanol, and recovering the ziprasidone base.

In another aspect, the present invention provides for a ziprasidone base hemihydrate.

In another aspect, the present invention provides for a ziprasidone base hemihydrate containing about 1.9% to about 2.5% water by Karl Fischer.

### BRIEF DESCRIPTION OF THE FIGURE

FIG. 1 is an X-Ray powder diffractogram of ziprasidone Base Form B2.

FIG. 2 is an X-Ray powder diffractogram of ziprasidone HCl Form A.

FIG. 3 is an FTIR spectrum of ziprasidone HCl Form A.

FIG. 4 is an X-Ray powder diffractogram of ziprasidone HCl Form J.

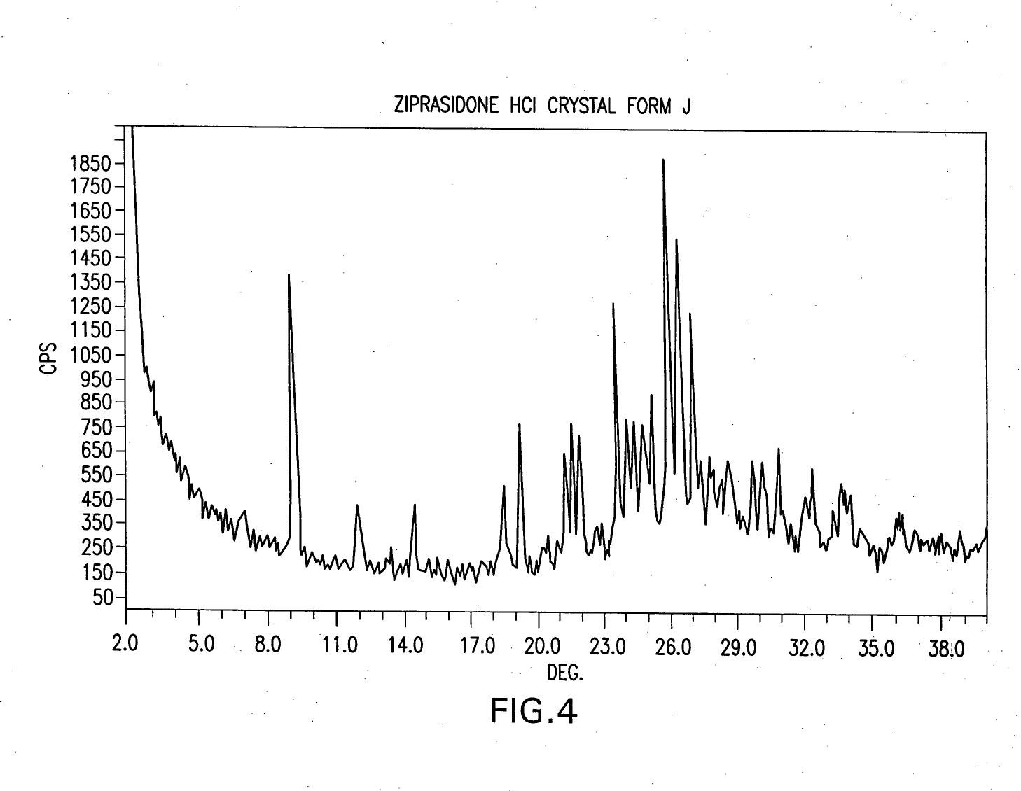

### DETAILED DESCRIPTION OF THE INVENTION

The present invention provides a crystal form, “Form B2”, of 5-[2-[4-(1,2-benzisothiazol-3-yl)-1-piperazinyl]ethyl]-6-chloro-1,3-dihydro-2 H -indol-2-one (ziprasidone base). Ziprasidone base Form B2 allows for preparing pharmaceutically acceptable salts of ziprasidone, such as the HCl salt and the mesylate salts. Ziprasidone base Form B2 also is an -ideal starting material form for preparing ziprasidone base Form B.

Ziprasidone base Form B2 may be prepared by reaction of a ziprasidone salt, most preferably the HCl salt, with a base in a reaction mixture containing water. Other salts such as acetic, benzoic, fumaric, maleic, citric, tartaric, gentisic, methane-sulfonic, ethanesulfonic, benzenesulfonic and laurylsulfonic, taurocholate and hydrobromide salts may be used Suitable bases for neutralization include, for example, an organic amine, an alkoxide, an alkali metal hydroxide, an alkaline earth metal hydroxide, an alkali metal hydride, an alkaline earth metal hydride or an alkali or alkaline earth metal carbonate or hydrogencarbonate salt. Specific examples of bases include, for example, 1,8-bis(N,N-dimethylamino)napthalene, sodium methoxide, sodium ethoxide, sodium phenoxide, sodium hydroxide, potassium hydroxide, calcium hydroxide, magnesium hydroxide, sodium hydride, potassium hydride, calcium hydride, sodium carbonate, potassium carbonate, sodium hydrogencarbonate, potassium hydrogencarbonate, calcium carbonate and basic alumina.

The reaction may be carried out without complete dissolution in a slurry or in a solution. When the reaction is carried out in water, a slurry is formed. It is possible to add an organic co-solvent to the water to increase the solubility of the solute, and thus form a solution. Examples of co-solvents include water miscible solvents such as a C₁ to C₄ alcohol (preferably methanol or ethanol) or tetrahydrofuran.

The reaction mixture (slurry or solution) may be heated. Preferably the reaction mixture is heated to a temperature of about 40 C to about reflux temperature. The amount of base used is preferably a molar excess sufficient to neutralize all of the salt. A preferred pH for the reaction is from 7 to about 10. The reaction is carried out for a sufficient time to neutralize all the salt, preferably for one hour at elevated temperature.

The base may then be recovered from the slurry or the solution by conventional techniques such as filtration, decanting, centrifugation, etc.

The ziprasidone base may be slurried for additional time in a solvent such as a C₁ to C₄ alcohol to further purify the recovered crystalline form. In a preferred embodiment, the base is slurried in iso-propanol to further increase the purity profile of the base.

The wet product may be dried under ambient or reduced pressure (less than about 50 mmHg). The temperature may be increased to preferably from about 40 EC to about 60 C to accelerate the drying process.

In one embodiment illustrated in the example, water, sodium carbonate and ziprasidone HCl are combined. The resulting heterogeneous mixture (slurry) is heated at elevated temperature for one hour, followed by filtration. With a slurry, the slurry is maintained for a sufficient time to obtain a conversion. Optimum time of conversion may be deciphered in routine nature by taking a sample from the slurry at various times.

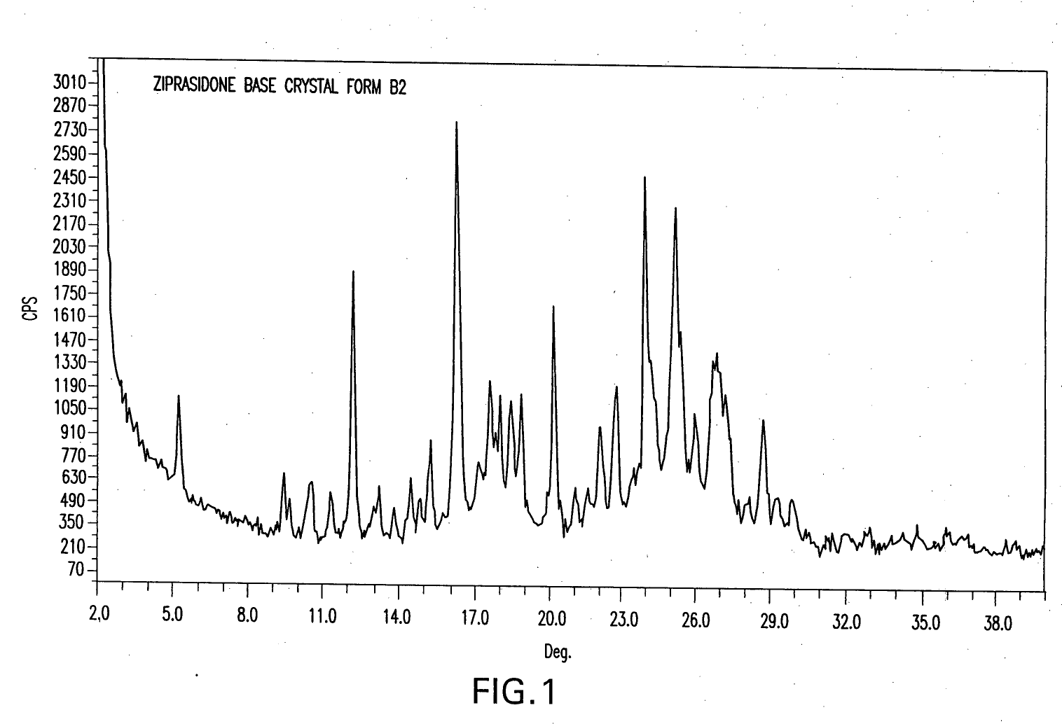

The X-Ray powder diffraction of ziprasidone base Form B2 (FIG. 1) obtained by neutralization of the HCl salt is characterized by peaks at 9.4, 13.7, 14.5, 14.9, 18.1, 20.2, 22.8±0.2 degrees 2 theta. Ziprasidone base form B2 contains about 1.9 to about 2.5% water according to Karl Fisher analysis. The water content points to a hemihydrate form.

Ziprasidone base Form B2 is useful inter alia as an intermediate for preparation of ziprasidone HCl salt or ziprasidone mesylate salt crystalline or amorphous, such as for preparation of ziprasidone HCl form A and ziprasidone HCl monohydrate of U.S. Pat. No. 5,312,925. Other polymorphic forms such as E, F, G, I, amorphous form, Form J, Form E1 and M may also be prepared. Ziprasidone HCl forms A, E, F, G, I and M are disclosed in U.S. provisional application No. 60/494,970, filed on Aug. 13, 2003, incorporated herein by reference. Other pharmaceutically acceptable salts of ziprasidone may also be prepared from ziprasidone base: acetic, benzoic, fumaric, maleic, citric, tartaric, gentisic, methane-sulfonic, ethanesulfonic, benzenesulfonic and laurylsulfonic, taurocholate and hydrobromide salts. Preferred salts are the hydrochloride and the mesylate. These pharmaceutically acceptable salts may be formulated for administration to a mammal, via the same route as GEODEN.

Preparation of ziprasidone HCl Form A from ziprasidone base Form B2 is illustrated in example 2. In this embodiment, HCl is added to a slurry of ziprasidone base in a mixture of water and a water miscible solvent, preferably a C₁ to C₃ alcohol, more preferably isopropanol. The reaction may be carried out at lower temperatures since acidification results in a temperature increase. In one embodiment, the reaction is carried out below room temperature, more preferably below about 10 C. Preferably, the reaction temperature is kept substantially constant.

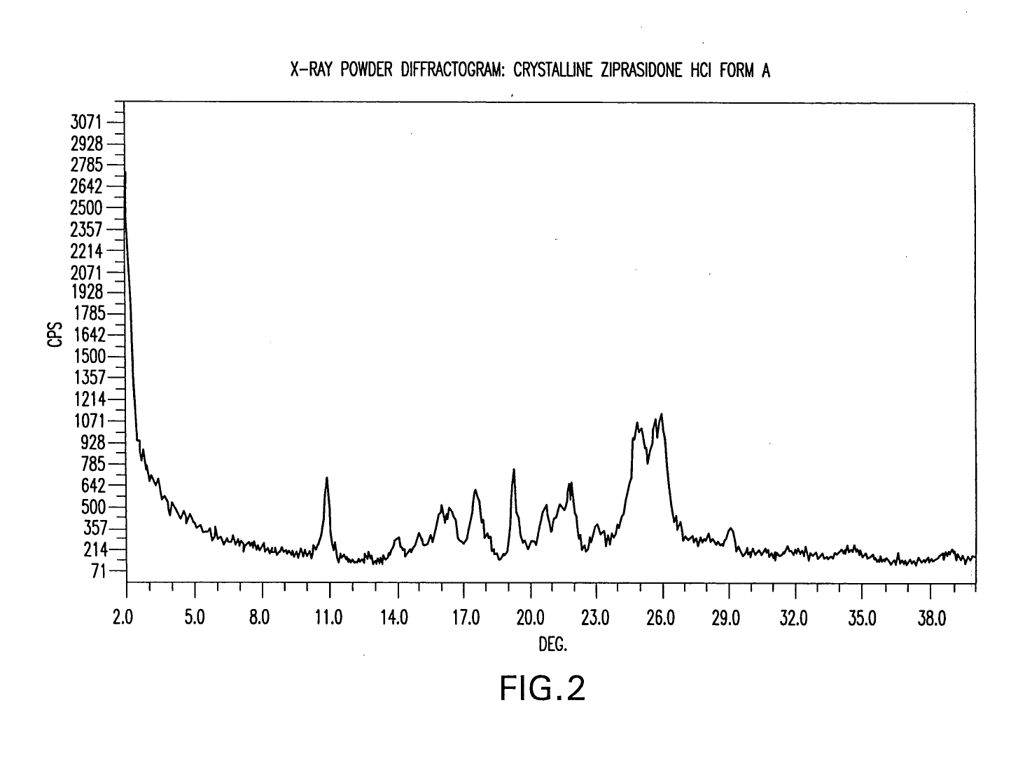

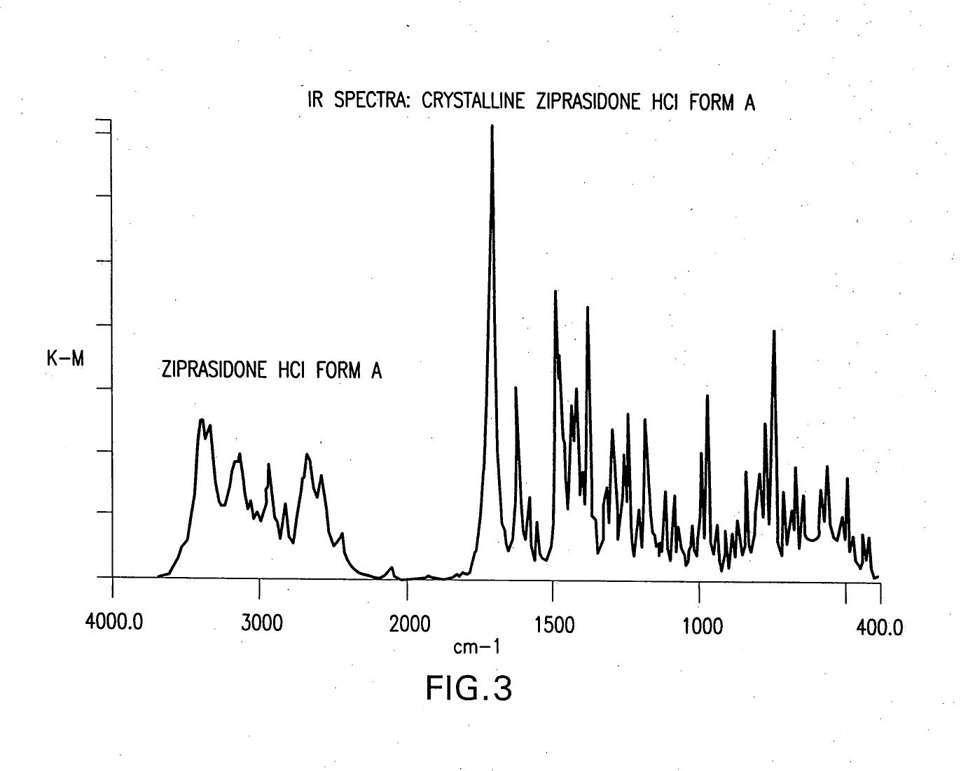

Ziprasidone HCl, denominated Form A, is characterized by data selected from the group consisting of an X-Ray diffraction pattern having peaks at about 10.9, 17.4 and 19.1±0.2 degrees 2 theta, substantially as depicted in FIG. 2, and an FTIR spectrum with characteristic absorption bands at about 3400, 3344, 3172, 2949, 970, 940, 872 and 843 cm⁻¹, substantially as depicted in FIG. 3. Crystalline ziprasidone HCl Form A may be further characterized by XRD peaks at 25.0 and 26.0±0.2 degrees two-theta, and may be further characterized by XRD peaks at 13.9, 20.6, 21.3, 21.8 and 23.0±0.2 degrees two-theta.

Ziprasidone base Form B2 may also be used to prepare ziprasidone HCl Form M (monohydrate). Form M may be prepared by adding HCl to a solution made from ziprasidone base B2 a solvent to precipitate Form M. Suitable solvents include THF, methanol, DMA, acetic acid and mixtures thereof. The temperature during addition of HCl is preferably above about 40 EC, more preferably above about 50 C.

Ziprasidone base Form B2 may also be used to prepare ziprasidone HCl hemihydrate by adding HCl solution to a slurry made from ziprasidone base B2 in C₂ to C₄ alcohol, preferably ethanol at elevated temperature, preferably above about 40 C, more preferably above about 50 C. Slurrying for about 4 hours to about 24 hours is sufficient.

The hemihydrate may be converted to ziprasidone HCl Form M by slurry in water at elevated temperature, preferably above about 40 C, more preferably above about 50 C.

Ziprasidone base Form B2 may also be used to prepare ziprasidone HCl anhydrous. By anhydrous it is meant lack of bound solvent, i.e., a solvent is not part of the crystal structure. Ziprasidone HCl anhydrous may be prepared by adding HCl to a slurry of ziprasidone base Form B2 in methanol. A C₁-C₄ alcohol with gaseous alcohol may be used. The reaction may be carried out substantially at room temperature, though optimization may be possible at other temperatures.

Ziprasidone base Form B2 may also be used to prepare ziprasidone HCl Form J. Ziprasidone HCl Form J may be prepared by adding HCl solution to a slurry made from ziprasidone base Form B2 in a C₅ to C₁₂ aromatic or aliphatic hydrocarbon, preferably toluene, heptane or hexane (straight or cyclic).

Crystalline ziprasidone HCl (Form J) is characterized by a powder XRD pattern with peaks at 9.1, 19.1, 25.7, 26.3, 26.9±0.2 degrees 2 theta.

Ziprasidone Form B2 also allows for preparation of other polymorphic forms of ziprasidone base. Form B2 may be slurried in an aprotic solvent such as a C₅ to C₁₂ hydrocarbon to obtain ziprasidone base Form B. Preferably, the slurry is at a temperature of at least about 60 C. Preferably, the hydrocarbon is toluene. In addition to toluene, other aprotic solvents may be used for the slurry, such as acetonitrile or dimethyl formamide (DMF). Ziprasidone base Form B may be recovered from the slurry by conventional techniques such as filtration.

Ziprasidone Form B2 may also be converted to ziprasidone base Form B by heating. In this embodiment, ziprasidone base Form B2 is heated to a temperature of at least about 50 EC, more preferably more than about 60 C. An ideal time for the slurry process is about 4 to about 24 hours. It is possible to use an air-circulated oven or reduced pressure during the heating.

In a preferred embodiment, ziprasidone base Form B is obtained by slurrying of the ziprasidone HCl in water in the presence of a base, followed by washing with methanol after recovering the obtained product, at a temperature of from about room temperature to about reflux temperature, and further heating at a temperature above about 30 C. Optionally, after the obtained product is recovered, the product is slurried and washed with C₁ to C₄ alcohols. Preferably, the alcohol is isopropanol.

Ziprasidone base Form B or another form of ziprasidone base may be converted to ziprasidone base Form B2 by precipitation. Ziprasidone base is dissolved in a suitable solvent and precipitated with an anti-solvent, preferably at elevated temperature. In one embodiment, ziprasidone base is dissolved in Tetrahydrofuran, and precipitated by addition of water. The temperature for addition on anti-solvent is preferably above about room temperature, more preferably above about 60 C.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

Pharmaceutical compositions of the present invention contain the above disclosed polymorphic forms of ziprasidone base or salts thereof (preferred salts hydrochloride and mesylate). In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose,-starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscamellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the active ingredient and any other solid excipients are suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The dosage of GEODON may be used as guidance. The oral dosage form of the present invention is preferably in the form of an oral capsule having a dosage of about 10 mg to about 160 mg, more preferably from about 20 mg to about 80 mg, and most preferably capsules of 20, 40, 60 and 80 mg. Another preferred dosage form is an injectable.

X-Ray powder diffraction data were obtained using by method known in the art using a SCINTAG powder X-Ray diffractometer model X'TRA equipped with a solid state detector. Copper radiation of 1.5418 Å was used. A round aluminum sample holder with round zero background quartz plate, with cavity of 25(diameter)*0.5(dept) mm. Detection limit: 5%.

IR analysis was done using a Perkin Elmer SPECTRUM ONE FT-IR spectrometer in DRIFTt mode. The samples in the 4000-400 cm⁻¹ interval were scanned 16 times with 4.0 cm⁻¹ resolution.

### EXAMPLE

### Example 1

### Preparation of Ziprasidone Base Crystal Form B2

In a 4 L three necked flask was charged 1 L water, 20 g Na₂CO₃ and 300 g ziprasidone HCl. To the obtained slurry, more water (1 l) and Na₂CO₃ (10 g) were added. The reaction mixture was heated at 60° C. and held for 1 hour. The solid was filtrated, washed with water (2×300 ml.), and ziprasidone base form B2 was obtained. In order to improve the chemical purity of the product, the wet solid was taken in isopropyl-alcohol (2 l) and the slurry was stirred at 60° C. for 2 hours; after cooling the solid was filtrated, washed with isopropyl-alcohol and dried at 50° C. for 23 hours. The solid after 23 h drying contained 2.3% water (by K.F.) and after 2 days drying contained 2.1% water (by K.F.). The XRD of the material after drying was that of ziprasidone base Form B2.

In this example the ziprasidone HCl used was Form A, but other forms of ziprasidone HCl may be used.

### Example 2

### Preparation of Ziprasidone HCl form A from Ziprasidone base B2

Into a 250 ml reactor were charged ziprasidone base form B2 (10 g), isopropyl alcohol (25 ml) and water (25ml). The obtained slurry was cooled to 5° C. HCl (32%, 29.4 ml) was added drop-wise over about 10 minutes. The temperature over the HCl addition was maintained below 10° C. The reaction mixture was stirred at this temperature for 24 hours, so that the solid was filtrated, washed with a mixture IPA/water 1:1 and dried in a vacuum oven at 50° C. The final material was ziprasidone HCl form A (KF 4.5%).

### Example 3

### Preparation of Ziprasidone base form B from form Ziprasidone Base Form B2

In a 0.5 l three necked flask was charged ziprasidone base (50 g) and toluene (250 ml), and the obtained slurry was heated at 85° C. for 2 hours. The hot slurry was filtrated and the solid was washed with methanol. The solid was dried in air-circulated oven at 50° C. to afford the dried ziprasidone base Form B (by XRD) (45.39 g).

### Example 4

### Preparation of Ziprasidone Base Form B

To the slurry of ziprasidone HCl form A (300 g) in 1 l water was added the solution of Na₂CO₃ (20 g) in 1 l water. The pH reached was 6.0. Additional amount of base was added (10 g) until the pH was 8 and the whole was heated at 60° C. for 1 h. After cooling the reaction mixture to the room temperature the solid was filtrated, washed with water (a sample was analyzed by XRD and the result indicates that was ziprasidone base form B2. After this the wet material was slurried in isopropyl alcohol (2 l) at 60° C. for 2 hours. The solid was filtrated and washed with IPA and then with methanol at room temperature. The wet material (ziprasidone base form B according to the XRD) was dried at 60° C. to afford the dried solid ziprasidone base form B (by XRD) (water content by K.F. 0.89%).

### Example 5

### Preparation of Ziprasidone Base Form B by Drying Ziprasidone Base Form B2

Ziprasidone base form B2 (20 g) was dried in a vacuum oven at 80° C. for 14 hours. The solid after drying was ziprasidone base form B.

### Example 6

### Preparation of Ziprasidone Base From B2

Ziprasidone base (30 g) was dissolved in a mixture THF/water 12.5:1 (1650 ml) by heating at reflux. To the solution active charcoal and Tonsil was added for color improvement. After 15 min. stirring, the mixture was filtrated and to the hot solution at about 60° C. water (1000 ml ) was added, than the solution was cooled to ˜2° C. After 2 hours the solid was filtrated, washed with mixture THF/water and dried at 40° C. to afford ziprasidone base cryst. (42.5 g). XRD of the sample indicates that was ziprasidone base form B2.

### Example 7

### Preparation of Ziprasidone HCl Form M From Ziprasidone Base Form B2

Into a flask were charged ziprasidone base form B2 (20 g) and 700 ml mixture THF:AcOH 9:1. Upon heating at 60° C. the whole came to a clear solution. Few drops of HCl 10% were added until turbidity was observed and than more HCl 10% (60 ml) was added slowly. The stirring was continued for 1 h and the heating source was removed. The solid was filtrated, washed with the same solvents mixture and dried at 50° C. for 1 hour and that was kept in a hood at the room temperature. The XRD indicates that the solid was ziprasidone HCl form M.

### Example 8

### Preparation of Ziprasidone HCl Form M From Ziprasidone Base Form B2

In a reactor was charged ziprasidone base form B2 (5 g), N,N-dimethylacetanide (DMA) (100 ML) and the mixture was heated at 60° C. To the obtained solution HCl was added (over 5 min.) and the stirring was continued at 60° C. for 4 hours. The solid obtained was filtrated, washed with DMA and dried over night in a vacuum oven at 50° C. The dried solid was ziprasidone HCl form M.

### Example 9

### Preparation of Ziprasidone HCl Form M From Ziprasidone Base Form B2 in THF/Methanol

Ziprasidone base form B2 (5 g) was dissolved almost completely in a mixture THF/MeOH 10:3 (225ml) at 60° C. Aqueous HCl 32% (20 ml) was added at this temperature during about 1 hour. The stirring was maintained at 60° C. over night. Than the slurry was cooled to the room temperature and the solid filtrated, washed with the same solvents mixture and dried at 50° C. The dried solid was ziprasidone HCl form M.

### Example 10

### Preparation of Ziprasidone HCl Hemihydrate From Ziprasidone Base Form B2

Into a flask were charged ziprasidone base form B2 (5 g ) and 150 ml abs. ethanol and the slurry was heated to 65° C. To the hot slurry a solution of aqueous HCl 32% (3 ml) in abs. Ethanol (50 ml) was drop-wise added during 1 hour and 30 min. The stirring was continued at this temperature over night. Part of the reaction mixture was filtrated while still hot and dried at 60° C. in a vacuum oven for 6 hours. The obtained solid was ziprasidone HCl hemihydrate.

The remained part of the reaction mixture was hold as follows: water (50 ml) was added to the hot slurry and the stirring was applied for additional 4 hours at 65° C. After this the solid was filtrated, dried in vacuum oven at 50° C. for 1.5 h and than in a fume hood for two days. This solid was ziprasidone HCl form M.

### Example 11

### Preparation of Ziprasidone HCl Anhydrous From Ziprasidone Base Form B2

To the slurry of ziprasidone base form B2 (10 g) in methanol (200 ml) at room temperature aqueous HCl 32% (10 ml) was added; over the HCl addition the temperature riched 30° C. The stirring was continued at room temperature for about 16 hours. The solid was filtrated, washed with methanol (2×10 ml) and dried at 60° C. The obtained solid was ziprasidone HCl anhydrous.

### Example 12

### Preparation of Ziprasidone HCl Form J From Ziprasidone Base Form B2

Into a flask were charged ziprasidone base form B2 (10 g) and toluene (200 ml); the slurry was agitated with mechanical stirrer. HCl 32% (20 ml) was added; a sticky material was formed. The solvent was removed by distillation and the dried solid was kept in cold in a closed container. The obtained solid was ziprasidone HCl form J.

### Example 13

### PROPHETIC—Preparation of Ziprasidone Mesylate Salt From Ziprasidone Base Form B2

Into a flask are charged ziprasidone base form B2 (10 g) and water (100 ml); the slurry is agitated with mechanical stirrer, methanesulfonic acid (2 ml) is added; the reaction mixture is heated to 60° C. for 4 hours, followed by cooling and filtratation. The obtained solid is ziprasidone mesylate salt.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used for guidance.

What is claimed is:

1. A process for preparing pharmaceutically acceptable salt of ziprasidone comprising:
a) reacting a salt of ziprasidone with a base, to obtain the crystalline form of ziprasidone base having an X-Ray powder diffraction pattern with peaks at 9.4, 13.7, 14.5, 14.9, 18.1, 20.2, 22.8±0.2 degrees 2 theta;
b) slurrying the crystalline form obtained in a C₁ to C₄ alcohol;
c) combining the crystalline form with an acid to obtain a pharmaceutically acceptable salt of ziprasidone; and
d) recovering the pharmaceutically acceptable salt.

2. The process of claim 1, wherein the acid is an HCl solution.
